(19) United States
(12) Patent Application Publication
(10) Pub. No.: US 2016/0249977 A1
(43) Pub. Date: Sep. 1, 2016

(54) RADIOFREQUENCY ABLATION ELECTRODE DEVICE HAVING DOUBLE-LAYERED UMBRELLA-SHAPED PROBES

(71) Applicant: MEDSPHERE INTERNATIONAL (SHANGHAI) CO., LTD., Shanghai (CN)

(72) Inventors: Bo Zhai, Shanghai (CN); Bo Xiao, Shanghai (CN); Jie Qin, Shanghai (CN); Weizhong Tang, Shanghai (CN)

(73) Assignee: MedSphere International (Shanghai) Co., Ltd., Shanghai (CN)

(21) Appl. No.: 15/028,422

(22) PCT Filed: Sep. 28, 2014
(86) PCT No.: PCT/CN2014/000880

§ 371 Date: Apr. 9, 2016

(30) Foreign Application Priority Data
Oct. 10, 2013 (CN) 201310470492.6
Oct. 10, 2013 (CN) 201320624128.6

(51) Int. Cl. A61B 18/14

(52) U.S. Cl. CPC A61B 18/1492; A61B 2018/00577

(57) ABSTRACT

A radio-frequency ablation electrode device with double-layered umbrella-shaped probes. The ablation electrode device comprises a temperature measuring tube (7) and a sleeve (5) sleeved outside of the temperature measuring tube (7). The head of the temperature measuring tube (7) is fixed to a first set of umbrella-shaped probes (8) in a welding mode, and a first fastening pipe (10) is fastened to the first set of probes (8) in a welding mode. The outside of the rear portion of the first set of probes (8) is coated with an insulating material layer, the outside of the insulating material layer is fixedly connected with a second set of umbrella-shaped probes (9), and a second fastening pipe (11) is fastened to the second set of probes (9) in a welding mode. The first set of probes (8), the human body and the second set of probes (9) form a radio-frequency current circuit. When the ablation electrode device is not used, the temperature measuring tube (7), the first set of probes (8) and the second set of probes (9) are contracted into the sleeve (5); when the ablation electrode device is used, the temperature measuring tube (7) protrudes out of the head of the sleeve (5), and the first set of probes (8) and the second set of probes (9) are unfolded to form an umbrella shape.

### BACKGROUND OF INVENTION

1. Field of the Invention

The present invention relates to a radiofrequency ablation electrode, in particular to a radiofrequency ablation electrode device having double-layered umbrella probes, belonging to the technical field of medical apparatus.

2. Background Art

Radiofrequency used in radiofrequency ablation is a high-frequency vibration having a frequency reaching hundreds of thousands per second. A human body is a complex structure composed of numerous organic and inorganic substances, and body fluid that contains a large number of dielectrics, such as ions, water and colloidal particles. The human body conducts current mainly relying on ion migration. Under the actions of a high-frequency alternating current, the directions of ion concentration changes vary with the directions of the currents, making positive and negative half-cycles to-and-fro changes. Under high-frequency oscillation, ions located between the two electrodes move quickly in the directions of the power lines, gradually changing from moving into a vibration (oscillation) state. Because various ions have different sizes, masses, charges and moving velocities, the ions rub against each other and collide with other micro particles to generate a biological thermal (heat) effect. Due to poor heat dissipation of tumors, the temperature of tumor tissues is higher than that of normal tissues nearby. In addition, cancer cells are sensitive to high heat. Thus, cancer cells can be killed without adverse effects. Therefore, radiofrequency ablation has the advantages of extra effect and slight damage, and has been applied to tumor therapy much widely at present.

The Chinese utility model patent Application No: 201120079822.5, entitled “Tower-type Multilayer Probe Radiofrequency Ablation Electrode Device for Therapy of Tumors of Liver” (Publication date: Nov. 23, 2011) discloses a radiofrequency ablation electrode device, which employs a multilayer tower-type probe structure serving to provide a larger probe stretching range to realize deep contact with tumors. However, because a current loop needs to be formed in radiofrequency ablation, a specific cathode plate is needed when such radiofrequency ablation electrode is used, and radiofrequency energy passes through such current loop of electrode-human body-cathode plate.

The radiofrequency ablation electrode, which needs to be matched with the cathode plate for use, has relatively high requirements on corollary equipment and is very inconvenient to use, and it is also not conformable for people because the cathode plate is padded under the human body.

### SUMMARY OF INVENTION

The technical problem to be solved by the present invention is that: the existing radiofrequency ablation electrode, which needs to be matched with the cathode plate for use, has relatively high requirements on corollary equipment and is very inconvenient to use, and it is also not conformable for people because the cathode plate is padded under the human body.

Embodiments of the present invention adopt the following technical solutions:

A radiofrequency ablation electrode device having double-layered umbrella probes comprises a temperature measuring tube 7 and a sleeve 5 which is sheathed outside the temperature measuring tube 7. The head of the temperature measuring tube 7 is pinpoint-shaped, fixed to a first group of umbrella-shaped probes 8 in a welding manner, and fastened to the first group of probes 8 in a welding manner by using a first fastening tube 10. An insulating material layer is coated on the outside of a section of probe tube 7 in the rear of the first group of probes 8, and the outside of the insulating material layer is fixedly connected with the second group of umbrella-shaped probe 9 and fastened to the second group of probes 9 in a welding manner by using a second fastening tube 11. When radiofrequency energy passes by, the first group of probes, the human body and the second group of probes form a current loop. When the radiofrequency ablation electrode device is not used, the temperature measuring tube 7, the first group of probes 8 and the second group of probes 9 are shrunk to the inside of the sleeve 5. When the radiofrequency ablation electrode device is used, the temperature measuring tube 7 is ejected from the head of the sleeve 5, and the first group of probes 8 and the second group of probes 9 are propped open to form an umbrella shape.

The technical solution has the following characteristics: a structure form of the double-layered umbrella-shaped probes is employed, and meanwhile, insulation is formed between the first group of probes and the second group of probes by disposing an insulating material layer, such that the first group of probes, the human body and the second group of probes are connected in series through radiofrequency energy to form a current loop, without a cathode plate.

In a further embodiment, a socket (4) may be disposed at the tail end of the radiofrequency ablation electrode device. The first group of probes 8 are connected with the socket 4 by wires, and the second group of probes 9 are also connected with the socket 4 by wires.

In a further embodiment, a temperature sensor, by which the temperature is measured, may be disposed inside the temperature measuring tube 7.

In a further embodiment, the first group of probes 8 and the second group of probes 9 may be uniform in shapes and sizes.

In a further embodiment, the radiofrequency ablation electrode device may also comprise a front rod 1, which is fixedly connected with the rear end of the sleeve 5, wherein a medicine injection hole la, which is communicated with the sleeve 5, is disposed in the front rod 1.

The present invention has one or more of the following beneficial effects:

The requirement on corollary equipment is reduced because a cathode plate does not need to be used, and a patient also feels more comfortable.

The radiofrequency ablation electrode device is simpler and more convenient to use and is not limited by fixing equipment when in use.

The probes are in more sufficient contact with the human body owing to the arrangement of the double-layered umbrella-shaped probes, and the radiofrequency ablation effect is better.

Other aspects and advantages of the invention will be apparent from the following description and the appended claims.

### BRIEF DESCRIPTION OF THE DRAWINGS

FIG. 1 shows a structural schematic drawing of a radiofrequency ablation electrode device introduced in the background art.

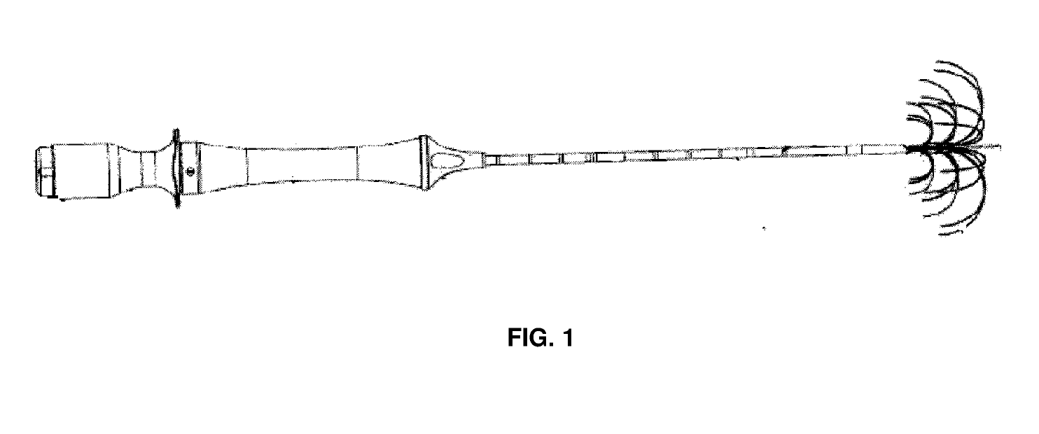

FIG. 2 shows a structural schematic drawing of a rear portion of a radiofrequency ablation electrode device in accordance with one embodiment of the present invention.

FIG. 3 shows a structural schematic drawing of a sleeve and a temperature measuring tube, with the sleeve being sheathed outside the temperature measuring tube.

FIG. 4 shows structural schematic drawing of a head of the radiofrequency ablation electrode device in accordance with one embodiment of the present invention.

In FIGS., front rod 1, medicine injecting hole la, hand lever 2, handle 3, socket 4, sleeve 5, medicine injecting hole plug 6, temperature measuring tube 7, first group of probes 8, second group of probes 9, first fastening tube 10 and second fastening tube 11 are marked.

### DETAILED DESCRIPTION OF THE INVENTION

The present invention is further illustrated below together with specific embodiments.

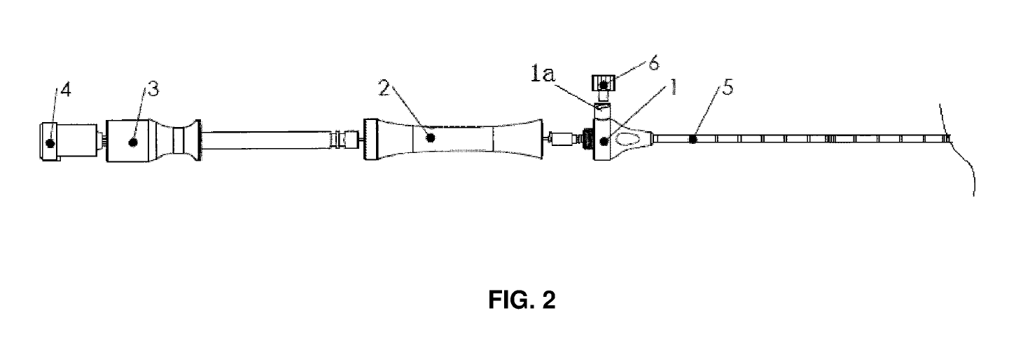

Referring to FIG. 2, a radiofrequency ablation electrode device having a double-layered umbrella-shaped probes in accordance with one embodiment of the present invention comprises a front rod 1, a hand lever 2, a handle 3, a socket 4, a sleeve 5, and other structures.

Referring to FIG.. 3, a temperature measuring tube 7 is a long tube inserted in the sleeve 5, wherein the head of the temperature measuring tube 7 is pinpoint-shaped and is internally provided with a temperature sensor (thermocouple) configured to measure a temperature.

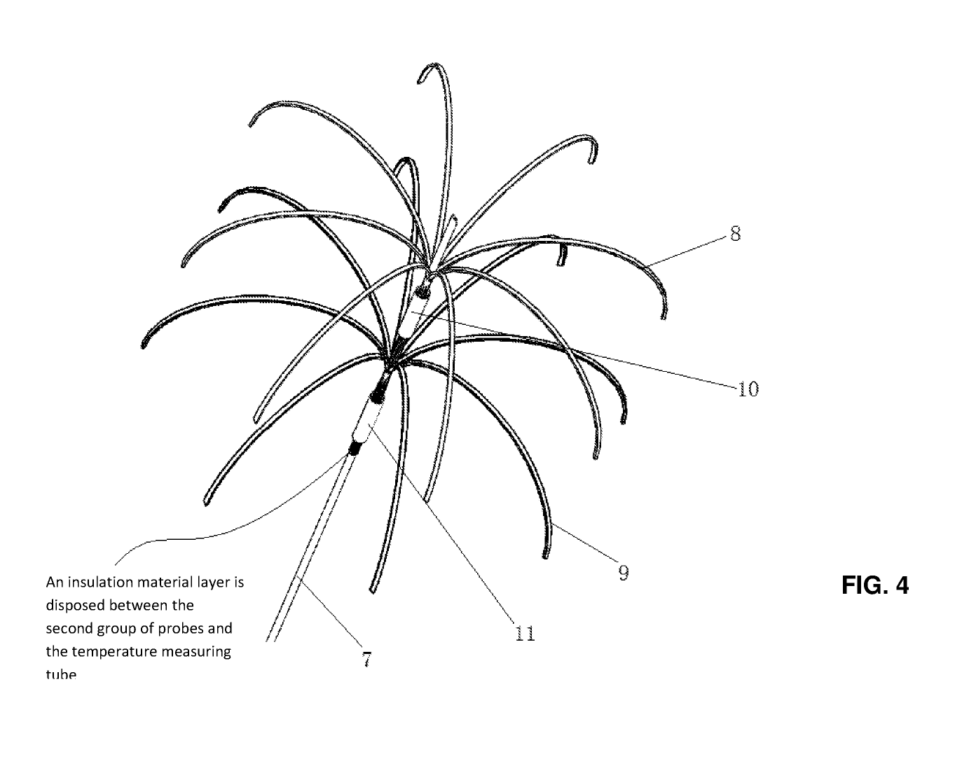

Referring to FIG. 4, the head of the temperature measuring tube 7 is pinpoint-shaped, fixed to a first group of umbrella-shaped probes 8 in a welding manner—e.g., fastened to the first group of probes 8 in a welding manner using a first fastening tube 10. An insulating material layer is coated on the outside of the section of probe tube 7 that is behind the first group of probes 8. The outside of the insulating material layer is fixedly connected with the second group of umbrella-shaped probes 9, e.g., fastened to the second group of probes 9 in a welding manner using a second fastening tube 11.

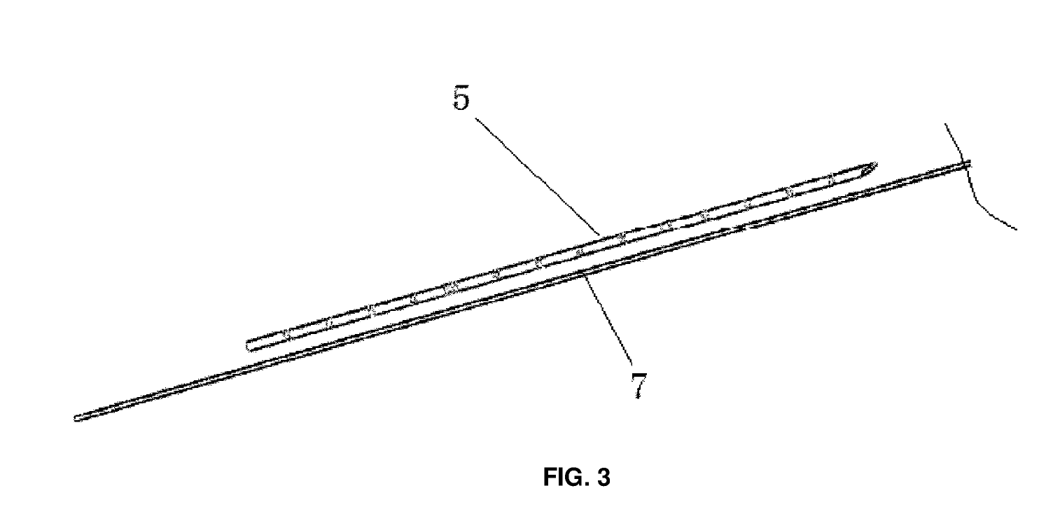

By referring to FIG. 2 to FIG. 4, a socket 4 is connected with an external power supply. The first group of probes 8 are connected with the socket 4 by a conductive wire, and the second group of probes 9 are also connected with the socket 4 by a conductive wire. When radiofrequency energy passes through the first groups of probes, the human body, and the second group of probes, it will form a current loop.

The first group of probes 8 and the second group of probes 9 have substantially the same shapes and sizes.

A radiofrequency ablation electrode device may further comprise a front rod 1 which is fixedly connected with the rear end of the sleeve 5, wherein a medicine injecting hole la, which is in communication with the sleeve 5, is disposed on the front rod 1.

When the radiofrequency ablation electrode device is not in use, the temperature measuring tube 7, the first group of probes 8, and the second group of probes 9 are shrunk (collapsed) to the inside of the sleeve 5. When the radiofrequency ablation electrode device is in use, the temperature measuring tube 7 is ejected from the head of the sleeve 5, and the first group of probes 8 and the second group of probes 9 are propped open to form an umbrella shape. A medicinal solution, when injected from the medicine injecting hole la, may be injected into the human body along the sleeve 5.

A structure form of a double-layered umbrella-shaped probes is employed in the embodiments of the present invention. Meanwhile, an insulation is formed between the first group of probes and the second group of probes by arranging an insulating material layer, such that the first group of probes, the human body, and the second group of probes can be connected in series to form a current loop, without a cathode plate.

What is claimed is:

1. A radiofrequency ablation electrode device having double-layered umbrella probes, characterized in that the device comprises a temperature measuring tube (7) and a sleeve (5),
wherein the sleeve (5) is sheathed outside the temperature measuring tube (7),
wherein a head of the temperature measuring tube (7) is pinpoint-shaped and is fixed, using a first fastening tube (10) in a welding manner, to a first group of umbrella-shaped probes (8),
wherein an insulating material layer is coated on an outside of a section of probe tube (7) located behind the first group of probes (8),
wherein an outside of the insulating material layer is fixedly connected, using a second fastening tube (11) in a welding manner, with a second group of umbrella-shaped probe (9), such that radiofrequency energy passes through the first group of umbrella-shaped probes, the human body, and the second group of umbrella-shaped probes forms a current loop,
wherein when the radiofrequency ablation electrode device is not in use, the temperature measuring tube (7), the first group of umbrella-shaped probes (8) and the second group of umbrella-shaped probes (9) are shrunk to an inside of the sleeve (5), and when the radiofrequency ablation electrode device is in use, the temperature measuring tube (7) is ejected from the head of the sleeve (5), and the first group of umbrella-shaped probes (8) and the second group of umbrella-shaped probes (9) are propped open to form an umbrella shape.

2. The radiofrequency ablation electrode device according to claim 1, wherein a socket (4) is disposed at a tail end of the radiofrequency ablation electrode device, the first group of umbrella-shaped probes (8) are connected with the socket (4) via a conductive wire, and the second group of umbrella-shaped probes (9) are also connected with the socket (4) via a conductive wire.

3. The radiofrequency ablation electrode device according to claim 1, wherein a temperature sensor, by which a temperature is measured, is disposed inside the temperature measuring tube (7).

4. The radiofrequency ablation electrode device according to claim 1, wherein the first group of umbrella-shaped probes (8) and the second group of umbrella-shaped probes (9) have substantially the same shapes and sizes.

5. The radiofrequency ablation electrode device according to claim 1, further comprising a front rod (1), which is fixedly connected with the rear end of the sleeve (5), wherein a medicine injection hole (la), which is in communication with the sleeve (5), is disposed on the front rod (1).
